(19) United States
(12) Patent Application Publication
(10) Pub. No.: US 2025/0281603 A1
(43) Pub. Date: Sep. 11, 2025

(54) Compositions for the Treatment of Infectious Disease Related Complications and Methods Thereof

(71) Applicant: Amarantus Bioscience Holdings, New York, NY (US)

(72) Inventor: Gerald COMMISSIONG, New York, NY (US)

(21) Appl. No.: 18/928,386

(22) Filed: Oct. 28, 2024

Related U.S. Application Data
(63) Continuation of application No. PCT/US2023/066317, filed on Apr. 27, 2023.
(60) Provisional application No. 63/335,351, filed on Apr. 27, 2022.

(51) Int. Cl. A61K 39/215; A61K 38/00; A61K 39/00; C07K 14/47; C12N 15/86

(52) U.S. Cl. CPC A61K 39/215; C07K 14/4702; C12N 15/86; A61K 38/00; A61K 2039/54; C07K 2319/40; C12N 2740/15043; C12N 2750/14043

(57) ABSTRACT

Provided herein are compositions and methods for the treatment of infectious diseases including COVID and Long COVID. The compositions and methods can be used to treat symptoms relating to infectious disease infection or vaccination as well as for immune system response enhancement to infection and vaccination. A composition includes Mesencephalic Astrocyte-derived Neurotrophic Factor (MANF) proteins and/or nucleic acids or functional fragments thereof. A method includes the treatment of infectious diseases such as COVID and long COVID, and for enhancement of immunogenic response to infectious diseases using the compositions disclosed herein.

### CROSS REFERENCE TO RELATED APPLICATIONS

This application is a continuation of PCT/US2023/066317, filed Apr. 27, 2023, which claims priority under 35 U.S.C. § 119 from Provisional Application Ser. No. 63/335,351, filed Apr. 27, 2022, the disclosures of which are incorporated herein by reference in their entirety.

### SEQUENCE LISTING

The instant application contains a Sequence Listing which has been submitted electronically in ST.26 (xml) format and is hereby incorporated by reference in its entirety. Said ST.26 (xml) copy, created on Mar. 7, 2024, is named 217640-701601_xml and is 4,160 bytes in size.

### FIELD OF THE INVENTION

The present disclosure relates to the field of infectious diseases. More specifically, the present disclosure relates to compositions and methods for the treatment of complications arising from an infection with an infectious disease or from the vaccination to an infectious disease.

### SUMMARY OF THE DISCLOSURE

The present disclosure relates to an engineered Mesencephalic Astrocyte-derived Neurotrophic Factor (MANF) protein or a functional fragment thereof or a nucleic acid encoding the engineered MANF protein or the functional fragment thereof, compositions thereof, and methods of treating conditions arising from infection with an infectious disease or vaccination to the infectious disease.

Provided herein are engineered MANF proteins or functional fragments thereof or a nucleic acid encoding the engineered MANF protein or the functional fragment thereof. In some embodiments, the engineered MANF protein or the functional fragment thereof has at least about 80% identity to SEQ ID NO: 2. In some embodiments, the engineered MANF protein or the functional fragment thereof has at least about 85% identity to SEQ ID NO: 2. In some embodiments, the engineered MANF protein or the functional fragment thereof has at least about 90% identity to SEQ ID NO: 2. In some embodiments, the engineered MANF protein or the functional fragment thereof has at least about 95% identity to SEQ ID NO: 2. In some embodiments, the engineered MANF protein or the functional fragment thereof has at least about 98% identity to SEQ ID NO: 2. In some embodiments, the engineered MANF protein or the functional fragment thereof is identical to SEQ ID NO: 2. In some embodiments, the nucleic acid encoding the engineered MANF protein or the functional fragment thereof has at least about 80% identity to SEQ ID NO: 1. In some embodiments, the nucleic acid encoding the engineered MANF protein or the functional fragment thereof has at least about 85% identity to SEQ ID NO: 1. In some embodiments, the nucleic acid encoding the engineered MANF protein or the functional fragment thereof has at least about 90% identity to SEQ ID NO: 1. In some embodiments, the nucleic acid encoding the engineered MANF protein or the functional fragment thereof has at least about 95% identity to SEQ ID NO: 1. In some embodiments, the nucleic acid encoding the engineered MANF protein or the functional fragment thereof has at least about 98% identity to SEQ ID NO: 1. In some embodiments, the nucleic acid encoding the engineered MANF protein or the functional fragment thereof is identical to SEQ ID NO: 1.

```
SEQ ID NO: 1
      1 cttcggtcct gctgtagtgc cttctgcgcc aggcccggtt caatcagcgg ccacaactgt
    61 ctagggctca gacaccacca gccaatgagg gagggcacgt ggagccgcgt ctgggctcgc
  121 ggctcctgac caatggggaa gtggcatgtg ggagggcgcc ggggttcccc ccgccaatgg
  181 ggagctacgg cgcgcggccg ggacttggag gcggtgcggc gcggcgggtg cggttcagtc
  241 ggtcggcggc ggcagcggag gaggaggagg aggaggagga tgaggaggat gaggaggatg
  301 tgggccacgc aggggctggc ggtgcgcgtg gctctgagcg tgctgccggg cagccgggcg
  361 ctgcggccgg gcgactgcga agtttgtatt tcttatctgg gaagatttta ccaggacctc
  421 aaagacagag atgtcacatt ctcaccagcc actattgaaa acgaacttat aaagttctgc
  481 cgggaagcaa gaggcaaaga gaatcggttg tgctactata tcggggccac agatgatgca
  541 gccaccaaaa tcatcaatga ggtatcaaag cctctggccc accacatccc tgtggagaag
  601 atctgtgaga agcttaagaa gaaggacagc cagatatgtg agcttaagta tgacaagcag
  661 atcgacctga gcacagtgga cctgaagaag ctccgagtta aagagctgaa gaagattctg
  721 gatgactggg gggagacatg caaaggctgt gcagaaaagt ctgactacat ccggaagata
  781 aatgaactga tgcctaaata tgcccccaag gcagccagtg caccgaccga tttgtagtct
  841 gctcaatctc tgttgcacct gagggggaaa aaacagttca actgcttact cccaaaacag
  901 cctttttgta atttattttt taagtgggct cctgacaata ctgtatcaga tgtgaagcct
  961 ggagctttcc tgatgatgct ggccctacag tacccccatg aggggattcc cttccttctg
1021 ttgctggtgt actctaggac ttcaaagtgt gtctgggatt tttttattaa agaaaaaaaa
1081 tttctagctg tcaaaaaaaa aaa
SEQ ID NO: 2
      1 MGKWHVGGRR GSPRQWGATA RGRDLEAVRR GGCGSVGRRR QRRRRRRRRM RRMRRMWATQ
    61 GLAVRVALSV LPGSRALRPG DCEVCISYLG RFYQDLKDRD VTFSPATIEN ELIKFCREAR
  121 GKENRLCYYI GATDDAATKI INEVSKPLAH HIPVEKICEK LKKKDSQICE LKYDKQIDLS
  181 TVDLKKLRVK ELKKILDDWG ETCKGCAEKS DYIRKINELM PKYAPKAASA PTDL
```

A sequence disclosed herein can individually and independently comprise a sequence with at least about: 10%, 15%, 20%, 25%, 30%, 35%, 40%, 45%, 50%, 55%, 60%, 65%, 70%, 75%, 80%, 85%, 90%, 95%, 100% sequence length (percent length) to any disclosed sequence herein. For example, a sequence can comprise a sequence with at least about: 10%, 15%, 20%, 25%, 30%, 35%, 40%, 45%, 50%, 55%, 60%, 65%, 70%, 75%, 80%, 85%, 90%, 95%, 100% sequence length to Seq ID No: 1 or Seq ID No: 2.

In some embodiments, the nucleic acid encoding the engineered MANF protein or the functional fragment thereof further comprises a modified nucleotide. In some embodiments, the modified nucleotide includes non-naturally occurring synthetic analogs of the nucleotides and the linkages between them, such as, for example and without limitation, a peptide nucleic acid, a morpholino, a phosphorothioate linkage, a locked nucleic acid, a glycol nucleic acid, an additional functional group comprising an amino (—NH2), fluoro (—F), or O-methyl (—OCH3) in the 2′-position of ribose sugar, a threose nucleic acid, a hexitol nucleic acid, a 2′-position sugar modification, a 5-position pyrimidine modification, an 8-position purine modification, a modification at exocyclic amine, a substitution of 4-thiouridine, a 5-((3-indolyl)propionamide-N-allyl)-20-deoxyuridine, a substitution of 5-bromo or 5-iodo-uracil as well as backbone modifications, an anti-reverse cap analog, a substitution of pseudouridine, a 5-methylcytidine and/or N1-methyluridine, or a methylation and unusual base-pairing combinations comprising the isobases isocytidine and isoguanidine and (7-(2-thienyl)imidazo[4,5-b]pyridine, Ds), and combinations thereof.

In some embodiments, the engineered MANF protein or the functional fragment thereof further comprises an epitope-tag fused at a C-terminus of the engineered MANF protein or functional fragment thereof or the nucleic acid encoding the engineered MANF protein or the functional fragment thereof further comprises a sequence encoding the epitope-tag. In some embodiments, the epitope-tag is an ALFA-tag, an AViTag, a C-tag, a Calmodulin-tag, an intein capture tag, a polyglutamate tag, a polyarginine tag, an E-tag, a FLAG-tag, an HA-tag, a His-tag, a Gly-His-tag, a Myc-tag, an NE-tag, a Rho1D4-tag, a S-tag, a SBP-tag, a softag 1, a softag 3, a spot-tag, a strep-tag, a T7-tag, a TC tag, a Ty tag, a V5 tag, a VSV-tag, or an Xpress tag.

Provided herein are vectors and plasmids comprising the nucleic acid encoding the engineered MANF protein or the functional fragment thereof. In some embodiments, the vector or plasmid is a viral vector. In some embodiments, the vector or plasmid is a lentiviral vector or an AAV vector.

Also provided herein are compositions comprising the engineered MANF protein or the functional fragment thereof or the nucleic acid encoding the engineered MANF protein or the functional fragment thereof or the vector or the plasmid comprising the nucleic acid and one or more adjuvants, excipients, carriers, and/or diluents.

Also provided herein are pharmaceutical compositions comprising the engineered MANF protein or the functional fragment thereof or the nucleic acid encoding the engineered MANF protein or the functional fragment thereof or the vector or the plasmid comprising the nucleic acid and one or more pharmaceutically acceptable adjuvants, pharmaceutically acceptable excipients, pharmaceutically acceptable carriers, and/or pharmaceutically acceptable diluents. In some embodiments, the pharmaceutical composition is in a unit dose form.

In some embodiments, the composition may include one or more inert excipients, which include water, buffered aqueous solutions, surfactants, volatile liquids, starches, polyols, granulating agents, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, disintegrating agents and the like. In some embodiments, buffered solutions are at physiological pH. In some embodiments, buffered solutions are buffered to the pH of the target tissue.

Provided herein are methods for treating a disease or a condition arising during or after an infection from an infectious disease or after a vaccination in a subject in need thereof, the method comprising administering a therapeutically effective amount of the engineered MANF protein or the functional fragment thereof or the nucleic acid encoding the engineered MANF protein or the functional fragment thereof; a vector or a plasmid; a composition; or a pharmaceutical composition disclosed herein.

In some embodiments, the step of administrating is by an intravenous injection, an intramuscular injection, a subcutaneous injection, or an intradermal injection.

In some embodiments, the infectious disease is a coronavirus. In some embodiments, the coronavirus is a SARS-COV-2 virus. In some embodiments, the SARS-COV-2 virus is a variant or mutant thereof. In some embodiments, the treatment increases a B-lymphocyte cell population in a subject having a SARS-COV-2 infection compared to a subject having a SARS-COV-2 infection without the treatment as measured by an in vitro or in vivo assay. In some embodiments, the in vitro assay is an ELISA or ELISpot Assay. In some embodiments, the in vitro assay is a flow cytometry, mass cytometry, or fluorescence microscopy assay. In some embodiments, the treatment reduces, ameliorates, or improves at least one condition arising during or after an infection from an infectious disease infection or after the vaccination. In some embodiments, the condition is one or more of long-term breathing problems, heart complications, a chronic kidney impairment, a stroke, a Guillain-Barre syndrome, a multisystem inflammatory syndrome, a neurological damage, a vascular damage, an organ damage, or a clotting.

These elements, together with the other aspects of the present disclosure and various features are pointed out with particularity in the claims annexed hereto and form a part of the present disclosure.

### DETAILED DESCRIPTION OF THE DISCLOSURE

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used herein is for the purpose of describing particular embodiments only and is not intended to be limiting of the disclosure. As used herein, the term “and/or” can include any and all combinations of one or more of the associated listed items. As used herein, the singular forms “a,” “an,” and “the” can include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. The terms “comprise(s),” “include(s),” “having,” “has,” “can,” “contain(s),” and variants thereof, as used herein, can be open-ended transitional phrases, terms, or words that do not preclude the possibility of additional acts or structures. All definitions contained herein are understood to include the plural form as well unless the context clearly dictates otherwise.

For the recitation of numeric ranges herein, each intervening number there between with the same degree of precision can be explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” can mean within an acceptable error range for the particular value as determined by one of ordinary skill in the art, which can depend in part on how the value is measured or determined, e.g., the limitations of the measurement system. For example, “about” means plus or minus 10%, per the practice in the art. Alternatively, “about” means a range of plus or minus 20%, plus or minus 10%, plus or minus 5%, or plus or minus 1% of a given value. Alternatively, particularly with respect to biological systems or processes, the term means within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

As used herein, the term “nucleic acid” can mean a linear polymer of nucleic acids. Said polymers can include promoter sequences that drive the expression of one or more genes of interest. As used herein, the term “nucleic acid construct” can include, but is not limited to, oligonucleotides, RNA, linear DNA, closed end linear DNA, ministrings, transposons, doggybone DNA, GenWand DNA, and minimalistic, immunologically defined gene expression (MIDGE) DNA. As used herein, a nucleic acid construct can be DNA or RNA or a mixture of the two and can comprise naturally occurring or artificial (non-natural) nucleotides. As used herein, “naturally occurring nucleotides” can include adenine, guanine, cytosine, thymine, uracil, inosine, 2,6-diaminopurine, 5-hydroxymethylcytosine, N4-methylated cytosine, N6-methylated adenine, archaeosine and other nucleotide modifications that occur in normal cellular (eukaryotic, prokaryotic or archaea, including virally-induced or phage-induced) metabolism. As used herein, “artificial nucleotide” or “non-natural nucleotide” is a nucleotide analog or linkage that is not naturally occurring and can include, but is not limited to, peptide nucleic acids, morpholinos, phosphorothioate linkages, locked nucleic acids, glycol nucleic acids, addition of functional groups such as amino (—NH2), fluoro (—F), and O-methyl (—OCH3) in the 2′-position of ribose sugar, threose nucleic acids, hexitol nucleic acids, 2′-position sugar modifications, 5-position pyrimidine modifications, 8-position purine modifications, modifications at exocyclic amines, substitution of 4-thiouridine, 5-((3-indolyl)propionamide-N-allyl)-20-deoxyuridine, substitution of 5-bromo or 5-iodo-uracil as well as backbone modifications, anti-reverse cap analogs, substitution of pseudouridine, 5-methylcytidine and/or N1-methyluridine, methylations and unusual base-pairing combinations including, but not limited to, the isobases isocytidine and isoguanidine and (7-(2-thienyl)imidazo[4,5-b]pyridine, Ds).

As used herein, the term “plasmid” can mean a circular DNA molecule that is physically separated from chromosomal DNA. As used herein, the term “plasmid” includes, but is not limited to, bacterially-derived plasmids, minicircles, episomal DNA, covalently closed circular DNA (cccDNA), extrachromosomal circular DNA (eccDNA), chromids, chloroplast DNA, baculovirus-derived circular DNA, bacmids, nanoplasmids, and mitochondrial DNA. The term “plasmid” includes, but is not limited to, small circular, double-stranded DNA molecules with promoter sequences that drive the transcription of one or more genes of interest. Optionally, plasmids can also include an origin of replication (ori) site, marker genes (e.g., antibiotic resistance genes) for selection and/or screening, enhancer elements and restriction endonuclease (RE) sites to allow inserts to be cloned in specific site. As used herein, a plasmid can comprise naturally occurring and/or artificial nucleotides.

In the context of the present disclosure, the terms “polypeptide” and “protein” are equivalent and mutually interchangeable. They refer to any amino acid chain, and include any post-translational modifications thereto (for example phosphorylation or glycosylation).

As used herein, the term “subject” or “patient” can refer to any organism to which a composition or formulation in accordance with the disclosure may be administered, e.g., for experimental, diagnostic, prophylactic, and/or therapeutic purposes. Typical subjects include animals (e.g., aquatic animals, land animals such as mice, rats, rabbits, non-human primates, and humans, fowl) and/or plants. In some embodiments, a subject is a human. Humans can be more than about: 1, 2, 5, 10, 20, 30, 40, 50, 60, 65, 70, 75, 80, 85, 90, 95, 100, 105, 110, 115 or about 120 years of age. Humans can be less than about: 1, 2, 5, 10, 20, 30, 40, 50, 60, 65, 70, 75, 80, 85, 90, 95, 100, 105, 110, 115 or about 120 years of age. In some cases, a human can be less than about 18 years of age. In some cases, human can be from about 1 week to about 5 weeks old, 1 month to about 12 months old, from about 1 year to about 20 years, from about 15 years to about 50 years, from about 40 years to about 80 years, or from about 60 years to about 110 years. In some cases, a human can be more than about 18 years of age. A human may be a pediatric subject. A human may be an adult subject. A human can be a child subject. A mammal such as a human can be born a male or a female.

As used herein, the term “administering” refers to providing a therapeutically effective amount of a chemical or biological compound or pharmaceutical composition to the subject, using intravitreal, intraocular, ocular, subretinal, intrathecal, intravenous, subcutaneous, transcutaneous, intracutaneous, intracranial, topical and the like administration. The chemical or biological compound of the present disclosure can be administered alone, but may be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; and the like.

The chemical or biological compound or pharmaceutical composition of the present disclosure may also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Specifically, the pharmaceutically acceptable carriers, excipients, binders, and fillers contemplated for use in the practice of the present disclosure are those which render the compounds of the disclosure amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. The compositions may include one or more inert excipients, which include water, buffered aqueous solutions, surfactants, volatile liquids, starches, polyols, granulating agents, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, disintegrating agents and the like. Buffered solutions will typically be at physiological pH and more particularly will typically be buffered to the pH of the target tissue.

The term “effective amount” or “therapeutically effective amount,” can mean to an amount of a composition or pharmaceutical composition as disclosed herein with or without additional agents that is effective to achieve its intended purpose, for example to treat a disease. Individual patient needs may vary. Generally, the dosage required to provide an effective amount of the composition will vary, depending on the age, health, physical condition, sex, weight, extent of the disease of the recipient, frequency of treatment and the nature and scope of the disease or condition. For example, a therapeutically effective amount of a composition of pharmaceutical composition herein can range from about 0.0001 mg/kg to about 10000 mg/kg, for example 0.001, 0.01 0.1, 1.0, 10.0 or 100.0, mg/kg, where mg is mg of composition or pharmaceutical composition and kg is kg of bodyweight of a subject or patient.

As used herein, the terms “treatment” or “treating” can mean a pharmaceutical or other intervention regimen for obtaining beneficial or desired results in the recipient. Beneficial or desired results include but are not limited to a therapeutic benefit and/or a prophylactic benefit. A therapeutic benefit refers to eradication or amelioration of one or more symptoms of an underlying disorder being treated. Also, a therapeutic benefit can be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For a prophylactic benefit, a subject at risk of developing a particular disease, or a subject reporting one or more of the physiological symptoms of a disease may undergo a treatment disclosed herein, even though a diagnosis of this disease may not have been made.

As used herein, “transfection” can refer to the introduction of a nucleic acid construct or plasmid or engineered DNA (e.g., a DNA) into a cell. Transfection can occur, for example, in vitro, ex vivo, or in vivo.

The present disclosure describes Mesencephalic Astrocyte-derived Neurotrophic Factor (MANF) engineered proteins and/or nucleic acids encoding the engineered proteins or a functional fragment thereof, vectors and plasmids, compositions and pharmaceutical compositions comprising said proteins for treatment of conditions including cardiovascular complications arising after an infectious disease infection or coronavirus (e.g., SARS-COV-2) infection. The engineered MANF proteins or compositions as disclosed herein are also used for treatment of damage to vital organs including retinal damage, damage to endothelial linings, and the like. The composition is also useful in enhancing the effectiveness of infectious disease (e.g., SARS-COV-2) vaccines and improve the immune response after the infectious disease (e.g., SARS-COV-2) infection.

In many cases SARS-COV-2 infections have reported cases of long-term organ damage. Although SARS-COV-2 infection is seen as a disease that primarily affects the lungs, it can also damage many other organs, including the heart, kidneys and the brain. Organ damage may lead to health complications that linger after illness. In some people, lasting health effects may include long-term breathing problems, heart complications, chronic kidney impairment, stroke and Guillain-Barre syndrome a condition that causes temporary paralysis and the like. Some adults and children experience multisystem inflammatory syndrome after they have had SARS-COV-2 infection. In this condition, some organs and tissues become severely inflamed.

Since the first pandemic wave of SARS-COV-2, scientists and doctors have observed neurological and vascular damage in infected people.

The compositions and methods of the present disclosure aim at providing treatment against the formation of clots formed due to clumping up of blood cells because of SARS-COV-2 infections. While large clots can cause heart attacks and strokes, much of the heart damage caused by COVID-19 is believed to stem from very small clots that block tiny blood vessels (capillaries) in the heart muscle. Other parts of the body affected by blood clots include the lungs, legs, liver and kidneys. COVID-19 can also weaken blood vessels and cause them to leak, which contributes to potentially long-lasting problems with the liver and kidneys.

The present disclosure further provides methods for enhancement of immune response elicited after an infectious disease (e.g., SARS-COV-2) infection or vaccination. The method leverages on the potential of MANF protein's ability to significantly enhanced β-cell proliferation leading to increase in B-lymphocyte population.

The method involves administering a therapeutically effective amount of Mesencephalic Astrocyte-derived Neurotrophic Factor (MANF) proteins and/or nucleic acid molecule, or a functional fragment thereof for treatment of any organ damage resulting because of SARS-COV-2 infections.

In some embodiments of the disclosure, MANF and/or biologically functional fragments thereof are administered to a subject to treat complications arising after SARS-COV-2 infection, including but not limited to complications arising out of damages to endothelial cells because of lack of oxygen across all vital organs.

In one aspect, the present disclosure relates to a composition comprising Mesencephalic Astrocyte-derived Neurotrophic Factor (MANF) proteins and/or nucleic acid molecule, or a functional fragment thereof for treatment of complications arising after SARS-COV-2 infection.

In another aspect, the present disclosure relates to a method for treatment of complications arising after SARS-COV-2 infection is provided. The method includes administering a therapeutically effective amount of a composition comprising a Mesencephalic Astrocyte-derived Neurotrophic Factor (MANF) proteins and/or nucleic acid molecule, or a functional fragment thereof.

In some embodiments, the step of administrating includes directly injecting said therapeutically effective amount of the composition comprising the MANF proteins or the functional fragment thereof to a subject having SARS-COV-2.

In some embodiments, the step of administrating includes managing β-cell proliferation leading to increase in B-lymphocyte population to enhance an immunological response of a subject having SARS-COV-2 infection.

In some embodiments, the pharmaceutical composition comprising Mesencephalic Astrocyte-derived Neurotrophic Factor (MANF) proteins and/or nucleic acid molecule or functional fragments thereof, may further contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates, other organic acids); bulking agents (such as mannitol or glycine), chelating agents [such as ethylenediamine tetraacetic acid (EDTA)]; complexing agents (such as caffeine, polyvinylpyrrolidone, beta-cyclodextrin or hydroxypropyl-beta-cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (preferably sodium or potassium chloride, mannitol sorbitol); delivery vehicles; diluents; excipients and/or pharmaceutical adjuvants.

In another aspect of the disclosure, Mesencephalic Astrocyte-derived Neurotrophic Factor (MANF) or one or more biologically functional fragments thereof are joined in a fusion through a linker or coupling agent. In some embodiments, the linker that can be used to conjugate MANF include, but are not limited to, hydrazones, thioethers, esters, disulfides and peptide-containing linkers. In some embodiments, the linker can be susceptible to cleavage by low pH or susceptible to cleavage by proteases, such as cathepsins (e.g., cathepsins B, C, D). For example, multiple peptides or polypeptides may be joined via a biologically-releasable bond, such as a selectively-cleavable linker or amino acid sequence.

In another aspect of the disclosure, the actual dosage amount of a composition of the present disclosure is administered to the subject can be determined by physical and physiological factors such as body weight, severity of condition, previous or concurrent therapeutic interventions, idiopathy of the patient and on the route of administration. The practitioner responsible for administration will, in any event, determine the concentration of active ingredient(s) in a composition and appropriate dose(s) for the individual subject.

### MANF

Mesencephalic astrocyte-derived neurotrophic factor (MANF), also known as arginine-rich mutated in early tumors, or ARMET, is a dopaminergic neurotrophic factor in astrocyte-conditioned medium. MANF is an ER soluble protein, localized to the luminal ER, that has cytoprotective effects, which are not limited to neurons.

MANF cDNA is encoded by a 4.3 kb gene with 4 exons and is located on human chromosome 3. The N-terminal 21 amino acids serve as the signal peptide, targeting nascent MANF to the BR. The secreted form of MANF is the full length protein without the signal sequence and has a molecular weight of 18 kDa. Nuclear magnetic resonance (NMR) spectroscopy of the three-dimensional solution structure of human MANF reveals that MANF is composed of an N-terminal saposin-like domain (residues 1-95) and a C-terminal SAP (SAF-A/B, Acinus and PIAS) domain (residues 104-158), which are connected with a short linker (residues 96-103). In some embodiments, the engineered MANF is the full protein. In some embodiments, the engineered MANF does not include the ER signal sequence.

MANF mRNA and protein are widely expressed in both neurons and nonneuronal tissues. In the brain, relatively high MANF levels are detected in the cerebral cortex, hippocampus and cerebellar Purkinje cells. In nonneuronal tissues, high MANF expression is detected in the adult liver, testis and salivary gland. In vitro, MANF expression is upregulated in cell lines derived from bone tissue (U2OS), kidneys (HEK293), neuroblastoma (SH-SY5Y) and embryo fibroblasts (NIH 3T3) in response to ER stress triggered by tunicamycin (TM), thapsigargin (TG), and lactatystin. In vivo, MANF expression is induced in chondrocytes following ER retention of the mutant cartilage extracellular matrix protein in mouse knock-in models of chondrodysplasia caused by matrilin-3 or type x collagen mutations, in pancreatic β cells following misfolding of mutant proinsulin in Akita mouse model carrying an insulin C96Y mutation, and in synoviocytes following inflammation mediated-ER stress in a rabbit antigen-induced arthritis model. MANF expression is also increased in response to ischemia-induced ER stress both in vitro and in vivo,

Related to ER stress-mediated kidney diseases, a podocyte ER stress-induced hereditary nephrotic syndrome (NS) mouse model, in which podocyte ER stress is activated by the C321R mutation of the glomerular basement membrane constituent laminin β2 (LAMB2), shows MANF is induced and secreted by ER-stressed podocytes. In acute kidney injury (AKI) triggered by ER stressor TM or ischemia/reperfusion (I/R), MANF is upregulated and secreted by ER-stressed tubules.

MANF expression, when compared with healthy controls, MANF mRNA is dramatically upregulated in peripheral white blood cells from patients with autoimmune inflammatory disease, including rheumatoid arthritis and systemic lupus erythematosus.

ER stress-induced transcriptional upregulation of MANF is driven by an ER stress response element (ERSE)-II in the MANF promoter. The trafficking and secretion of MANF is regulated by ER stress. Enhanced protein secretion is not a general cellular response to ER stress. However, a robust secretory response of MANF to TG-induced ER stress, but not to TM or DTT was observed in SH-SY5Y, cardiomyocytes and HeLa cells,

Brain ischemia induces neuron death through ER stress. It has been shown that in a rat model of focal cerebral ischemia induced by ipsilateral middle cerebral artery occlusion (MCAO), MANF protein is significantly induced in ischemic cortical neurons at the early stage after ischemia. An In vitro study showed that human recombinant MANF inhibits ER stress-induced apoptosis in cultured primary neurons. More importantly, in vivo studies demonstrate that intracerebral injection of human recombinant MANF 20 minutes before MCAO or intracortical delivery of AAV serotype 7 encoding human MANF one week before MCAO in rats reduces infarct volume and facilitates motor recovery. The neuroprotective effect of MANF is mediated, at least in part, through attenuation of ischemia-mediated cell apoptosis. In addition, ipsilateral ventricle injection of human recombinant MANF 2 hours after MCAO rescues the cerebral ischemia-induced neuron loss and promotes behavioral recovery in rats, which is associated with inhibition of ischemia-induced upregulation of ERSR proteins including BiP, phosphorylated IRE1 and XBP1s, as well as suppression of caspase 3 cleavage.

Myocardial infarction (MI) also activates ER stress in the surviving myocytes adjacent to the damaged region in vivo and simulated ischemia (sI) or simulated ischemia/reperfusion (sI/R) induces ERSR in cultured rat and mouse ventricular myocytes in vitro. Correlating with ER stress induction, MANF expression is shown to be upregulated in cardiac myocytes of mice subjected to MI in vivo and in cultured neonatal rat ventricular myocytes subjected to sI in an ATF6 and XBP1-dependent manner in vitro. miRNA-mediated knocking down of endogenous MANF in cultured cardiac myocytes increases cell death upon sI/R, whereas overexpression of MANF by adenovirus or addition of recombinant MANF to medium protects cardiac myocytes from serum starvation or sI- or sI/R-mediated cell death. Furthermore, infusion recombinant MANF into mice 24 hours prior to subjecting them to 30 minutes of myocardial ischemia has cardioprotective effects.

Immune modulation by MANF plays an important role in the repair and regeneration of retina. Regenerative therapies based on cell replacement hold promise for the treatment of a range of degenerative retina diseases, such as macular degeneration or retinitis pigmentosa. However, the proinflammatory microenvironments negatively affect integration and repair. A recent study has shown that in Drosophila or mouse, the damaged retina secretes Pvf-1 (platelet-derived growth factor (PDGF)-and vascular endothelial growth factor-related factor 1) and PDGF-A, respectively. These damage signals induce MANF expression in innate immune cells and MANF activation promotes a phenotype switch of macrophages from pro-inflammatory to anti-inflammatory, thereby improving tissue repair in both invertebrates and vertebrates, thus enhancing retinal regenerative therapy.

MANF can also repress proliferation of inflammatory fibroblast-like synoviocytes in a rodent methylated bovine serum albumin-induced arthritis model. Furthermore, MANF can alleviate oxygen-glucose deprivation-induced cell damage and inflammation in rat primary astrocytes, as well as suppress lipopolysaccharide-induced inflammatory response in neural stem cells.

Stress to the ER stimulates MANF expression and secretion. MANF regulates dopaminergic neuron development and MANF's deficiency may lead to diabetes and activation of the unfolded protein response. The mesencephalic astrocyte-derived neurotrophic factor (MANF) has been described as a survival factor for dopaminergic neurons in vitro, but its role in mammalian tissues is poorly understood.

SARS-COV-2 infection symptoms can in some cases be severe and persist for months. The virus is known to damage the lungs, heart, and/or brain, which increases the risk of long-term health problems. Most people who have SARS-COV-2 infection recover completely within a few weeks. But some people, even those who had mild versions of the disease, continue to experience symptoms after their initial recovery.

Most people who have SARS-COV-2 infection recover completely within a few weeks. But some people even those who had mild versions of the disease continue to experience symptoms after their initial recovery. These people sometimes describe themselves as “long haulers” and the conditions have been called post-COVID-19 syndrome or “long COVID-19.” These health issues are sometimes called post-COVID-19 conditions. They're generally considered to be effects of COVID-19 that persist for more than four weeks after you've been diagnosed with the COVID-19 virus. Older people and people with many serious medical conditions are the most likely to experience lingering COVID-19 symptoms, but even young, otherwise healthy people can feel unwell for weeks to months after infection. Common signs and symptoms that linger over time include Fatigue, Shortness of breath or difficulty breathing, Cough, Joint pain, Chest pain, Memory, concentration or sleep problems, Muscle pain or headache, Fast or pounding heartbeat, Loss of smell or taste, Depression or anxiety, Fever, Dizziness, Worsened symptoms after physical or mental activities.

Therefore, there is a need to provide effective therapies for treatment of SARS-COV-2 infection symptoms, cardiovascular complications arising out of the same, and compositions and methods to enhance the effectiveness of SARS-COV-2 Vaccines and improve the immune response after SARS-COV-2 infections.

Therefore, there is a need to provide effective therapies for treatment of SARS-COV-2 infection symptoms, cardiovascular complications arising out of the same, and compositions and methods to enhance the effectiveness of SARS-COV-2 Vaccines and improve the immune response after SARS-COV-2 infections.

### Nucleic Acid Constructs and Plasmids

Disclosed herein are nucleic acid constructs, vectors, and plasmids. In some embodiments, nucleic acid construct or a plasmid can comprise a polynucleotide, such as an engineered polynucleotide (e.g., engineered DNA) described herein. An engineered polynucleotide, such as engineered DNA can be described as a nucleic acid construct or a plasmid herein. In some embodiments, a vector can comprise a nucleic acid construct, a plasmid, an engineered polynucleotide, or any combination thereof disclosed herein. In some embodiments, an isolated cell can comprise a vector, nucleic acid construct, a plasmid, an engineered polynucleotide, or any combination thereof disclosed herein. In some cases, a cell can be an isolated and/or purified cell.

In some embodiments, the present disclosure can relate to a method of transferring a nucleic acid construct or plasmid of interest across the cell membrane. In some embodiments, the method can comprise: (a) adding one or more nucleic acid sequences encoding the engineered MANF protein or the functional fragment thereof sequences, and (b) introducing the nucleic acid, vector, or plasmid to the cytoplasm of a cell using one or more methods including, but not limited to, electroporation, iontophoresis, liposomal delivery, proteo-lipid vehicle delivery, extracellular vesicle delivery, endosomal delivery, polycation delivery, polyplex formation, chemical transfection, complexing with polyamines, nanoparticle delivery, inorganic nanocarrier delivery, metal organic nanocarrier delivery, microparticle delivery, detergent permeabilization, carbon nanotube delivery, viral delivery, virosome delivery, gene gun, jet-injector, biojector, laser-mediated phototransfection, biolistic particle delivery system, microneedles, microinjection, microfluidic squeezing, microfluidic vortex shedding, nanostraws, deterministic mechanoporation, polyethyleneimine delivery, and peptide conjugation.

In some embodiments, a plasmid encoding an engineered protein disclosed herein can be used in the manufacture of a viral vector. For example, one or more plasmids used in the manufacture of adeno-associated virus (AAV) for gene delivery can comprise an engineered protein as disclosed herein. A system for producing clinical grade AAV can be referred to as a helper-free system and can comprise three different plasmids encoding viral and helper genes: pHelper, AAV trans-plasmid comprising AAV replication (Rep) and capsid (Cap) genes, and AAV cis-plasmid encoding the gene of interest, promoter, and inverse terminal repeats (ITRs) that are used to transfect producer cell lines such as human embryonic kidney (HEK). Commercial production of AAV for clinical applications can require hundreds of grams if not kilograms of plasmid per year. Plasmids can also be used in the manufacture of other viral vectors including, but not limited to, retroviruses, adenoviruses and lentiviruses; inclusion of engineered MANF protein in the plasmids used for manufacturing these viral vectors can also greatly reduce the amount of plasmid required as well as the COGs for their manufacture. When using an AAV vector or AAV plasmid, the AAV can be, for example, AAV 1, AAV 2, AAV 3, AAV 4, AAV 5, AAV 6, AAV 7, AAV 8, or AAV 9.

In some embodiments, the methods described herein can be used for gene delivery in vivo. In some cases, a method can be used herein to deliver a gene to a subject in need thereof, for example a subject in need of an engineered protein to treat a disease condition. In some cases, a nucleic acid or plasmid can be relatively inexpensive to make. In some cases, a nucleic acid or plasmid can be more stable than a viral vector. In some cases, a viral vector can comprise a nucleic acid or plasmid. In some cases, a nucleic acid may not be limited by the size of the insert they can deliver. In some cases, a nucleic acid or plasmid may not contain viral capsids or envelope proteins. In some cases, a nucleic acid or plasmid may contain viral capsids or envelope proteins In some cases, a nucleic acid or plasmid may not invoke the type of immune response seen with viral vectors. In some embodiments, the disclosure can allow for the efficient use of nucleic acid or plasmids to deliver genes of interest to the nuclei of cells in vivo in a patient in need of treatment with or without the use of viral vectors. In some embodiments, the gene of interest can code for enzymes capable of modifying the DNA of a host cell. For example, such genes can include, but are not limited to, Crispr, Cas9, zinc finger nucleases, and TALENs.

In some embodiments, a method of treatment can comprise a therapeutically effective amount of a dose (e.g., a unit dose) of a nucleic acid construct or plasmid herein. In some cases, a composition disclosed herein can be administered to a subject in need of treatment in order to effect gene delivery. In some cases, a treatment may be preventive and/or therapeutic, and it may be directed to any disease or condition. In some cases, a condition or disease can comprise various genetic defects, but are not limited thereto. In some cases, a method can comprise a change of the genetic environment, e.g., by insertion of a plasmid containing a gene encoding a desired therapeutic function or by expressing an antigen that can induce a prophylactic or therapeutic immune response. In some cases, a treatment scheme can be determined by a physician in each case depending on such factors as the condition to be treated, age, and weight of the patient.

### Formulations and Excipients, Carriers and Diluents

In some embodiments, methods, composition, and systems disclosed herein can comprise pharmaceutical preparations. In some cases, a composition herein can comprise a pharmaceutical composition. In some cases, a pharmaceutical composition can be in unit dose form. In some cases, a formulation containing the compounds according to the present disclosure can take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

In some embodiments, a pharmaceutical composition can include a conventional pharmaceutical carrier or excipient and can additionally include other medicinal agents, carriers, adjuvants, additives and the like. In some cases, the composition can be about 0.1% to about 85%, about 0.5% to about 75% by weight of a compound or compounds of the disclosure, with the remainder consisting essentially of suitable pharmaceutical excipients.

In some embodiments, a composition herein can comprise an excipient. In some embodiments, a composition herein can comprise one or more of the following excipients: acacia, acesulfame potassium, acetic acid-glacial, acetone, acetyltributyl citrate, acetyltriethyl citrate, adipic acid, agar, albumin, alcohol, alginic acid, aliphatic polyesters, alitame, allantoin, almond oil, alpha hydroxy acids, alpha tocopherol, aluminum hydroxide adjuvant, aluminum monostearate, aluminum oxide, aluminum phosphate adjuvant, ammonia solution, ammonium alginate, ammonium chloride, argan oil, ascorbic acid, ascorbyl glucoside, ascorbyl palmitate, aspartame, attapulgite, azelaic acid, azulene, bakuchiol, beta glucan, beta-hydroxy-acids, bentonite, benzalkonium chloride, benzethonium chloride, benzoic acid, benzyl alcohol, benzyl benzoate, boric acid, bronopol, butylated glycol, butylated hydroxyanisole, butylated hydroxytoluene, butylene glycol, butylparaben, calcium acetate, calcium alginate, calcium carbonate, calcium chloride, calcium hydroxide, calcium lactate, calcium phosphate-dibasic anhydrous, calcium phosphate-dibasic dihydrate, calcium phosphate-tribasic, calcium silicate, calcium stearate, calcium sulfate, canola oil, capric glycol, capric triglyceride, carbomer, carbon dioxide, carboxymethylcellulose calcium, carboxymethylcellulose sodium, carrageenan, castor oil, castor oil-hydrogenated, cellulose-microcrystalline, cellulose-microcrystalline and carboxymethylcellulose sodium, cellulose-powdered, cellulose-silicified microcrystalline, cellulose acetate, cellulose acetate phthalate, ceramides, ceresin, cetostearyl alcohol, cetrimide, cetearyl alcohol, cetyl alcohol, cetylpyridinium chloride, chitosan, chlorhexidine, chlorobutanol, chlorocresol, chlorodifluoroethane (hcfc), chlorofluorocarbons (cfc), chloroxylenol, cholesterol, citric acid monohydrate, coconut oil, collagen, colloidal silicon dioxide, coloring agents, copper peptide, copovidone, corn oil, corn starch and pregelatinized starch, cottonseed oil, cresol, croscarmellose sodium, crospovidone, cyclodextrins, cyclomethicone, denatonium benzoate, desitin, dextrates, dextrin, dextrose, dibutyl phthalate, dibutyl sebacate, diethanolamine, diethyl phthalate, difluoroethane (hfc), dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol stearates, ethylene vinyl acetate, ethylparaben, fatty acids, ferulic acid, fructose, fumaric acid, gelatin, glucose-liquid, glycerin, glycerol, glyceryl behenate, glyceryl monooleate, glyceryl monostearate, glyceryl palmitostearate, glycine, glycofurol, glycolic acid, glycol stearate, guar gum, hectorite, heptafluoropropane (hfc), hexetidine, hydrocarbons (hc), hyaluronic acid, hydrochloric acid, hydrocortisone, hydrophobic colloidal silica, mesoporous silica, hydroquinone, hydroxyethyl cellulose, hydroxyethylmethyl cellulose, hydroxypropyl betadex, hydroxypropyl cellulose, hydroxypropyl cellulose-low-substituted, hydroxypropyl starch, hypromellose, hypromellose acetate succinate, hypromellose phthalate, imidurea, inulin, iron oxides, isomalt, isoparaffin, isopropyl alcohol, isopropyl myristate, isopropyl palmitate, jojoba oil, kaolin, kojic acid, lactic acid, lactitol, lactose-anhydrous, lactose-inhalation, lactose-monohydrate, lactose-monohydrate and corn starch, lactose-monohydrate and microcrystalline cellulose, lactose-monohydrate and povidone, lactose-monohydrate and powdered cellulose, lactose-spray-dried, lanolin, lanolin-hydrous, lanolin alcohols, lauric acid, lecithin, leucine, linoleic acid, macrogol 15 hydroxystearate, magnesium aluminum silicate, magnesium carbonate, magnesium oxide, magnesium silicate, magnesium stearate, magnesium trisilicate, maleic acid, malic acid, maltitol, maltitol solution, maltodextrin, maltol, maltose, mannitol, medium-chain triglycerides, meglumine, menthol, methionine, methylcellulose, methylparaben, mineral oil, mineral oil-light, mineral oil and lanolin alcohols, monoethanolamine, monosodium glutamate, monothioglycerol, myristic acid, myristyl alcohol, neohesperidin dihydrochalcone, neotame, niacinamide, nitrogen, nitrous oxide, octyldodecanol, oleic acid, oleyl alcohol, olive oil, palmitic acid, paraffin, peanut oil, pectin, PEG-8 stearate, pentetic acid, petrolatum, petrolatum and lanolin alcohols, phenol, phenoxyethanol, phenylethyl alcohol, phenylmercuric acetate, phenylmercuric borate, phenylmercuric nitrate, phospholipids, phosphoric acid, phytic acid, phytosphingosine, polacrilin potassium, poloxamer, polycarbophil, polydextrose, poly (dl-lactic acid), polyethylene glycol, polyethylene oxide, polymethacrylates, poly (methyl vinylether/maleic anhydride), polyoxyethylene alkyl ethers, polyoxyethylene castor oil derivatives, polyoxyethylene sorbitan fatty acid esters, polyoxyethylene stearates, polyoxylglycerides, polyparaben, polysorbate 60, polysorbate 80, polyvinyl acetate phthalate, polyvinyl alcohol, potassium alginate, potassium alum, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, propylparaben sodium, pyrrolidone, raffinose, Retin-A, retinol and retinoic acid derivatives, saccharin, saccharin sodium, safflower oil, salicylic acid, saponite, sesame oil, shellac, simethicone, sodium acetate, sodium alginate, sodium ascorbate, sodium benzoate, sodium bicarbonate, sodium borate, sodium carbonate, sodium chloride, sodium citrate dihydrate, sodium cyclamate, sodium formaldehyde sulfoxylate, sodium hyaluronate, sodium hydroxide, sodium lactate, sodium lauryl sulfate, sodium metabisulfite, sodium phosphate-dibasic, sodium phosphate-monobasic, sodium propionate, sodium starch glycolate, sodium stearyl fumarate, sodium ascorbyl phosphate, sodum deoxycholate, sodium hydroxide, sodium lauroyl lactylate, sodium lauryl sulfate, sodium palmitate, sorbitan stearate, sodium sulfite (E221), sprinolactone, sodium sulfite, sodium thiosulfate, sorbic acid, sorbitan esters (sorbitan fatty acid esters), sorbitan monostearate, sorbitol, soybean oil, sphingomyelins, starch, starch-pregelatinized, starch-sterilizable maize, stearic acid, stearyl alcohol, squalene, sucralose, sucrose, sucrose octaacetate, sugar-compressible, sugar-confectioner's, sugar spheres, sulfobutylether b-cyclodextrin, sulfur dioxide, sulfuric acid, sunflower oil, suppository bases-hard fat, tagatose, talc, tartaric acid, tetrafluoroethane (hfc), thaumatin, thimerosal, thymol, titanium dioxide, tragacanth, trehalose, tretinoin, triacetin, tributyl citrate, tricaprylin, triethanolamine, triethyl citrate, triethanolamine, triolein, undecylenic acid, vanillin, vegetable oil-hydrogenated, vitamins, vitamin e polyethylene glycol succinate, water, wax-anionic emulsifying, wax-carnauba, wax-cetyl esters, wax-microcrystalline, wax-nonionic emulsifying, wax-white, wax-yellow, xanthan gum, xylitol, zein, zinc acetate, and/or zinc stearate.

In some cases, a composition such as a pharmaceutical composition can comprise a carrier or a diluent. In some instances, a carrier or diluent can comprise a water, an alcohol, a salt solution (e.g., saline), or a mixture thereof. In some instances, a carrier can comprise a carbohydrate, a buffer, a salt, a pH adjuster, or any combination thereof. In some cases, a composition herein can comprise a buffering agent, a polymer, an antioxidant, a preservative, a chelating agent, a viscomodulator, a tonicifier, a flavorant, a colorant, an odorant, an opacifier, a suspending agent, a binder, a filler, a plasticizer, a lubricant, or any combination thereof.

In some embodiments, a liquid composition can be prepared by dissolving or dispersing an engineered MANF protein of functional fragment thereof, a nucleic acid, or plasmids disclosed herein in one or more of the above formulations (about 0.5% to about 20% by weight or more), and optional pharmaceutical adjuvants, in a carrier, such as, for example, aqueous saline, aqueous dextrose, glycerol, or ethanol, to form a solution or suspension. In some cases, for use in an oral liquid preparation, the composition can be prepared as a solution, suspension, emulsion, or syrup, being supplied either in liquid form or a dried form suitable for hydration in water or normal saline.

In some embodiments, an injectable composition for parenteral administration (e.g., intravenous, intramuscular, or intrathecal) can contain the compound in a suitable intravenous solution, such as sterile physiological salt solution. The composition can also be formulated as a suspension in an aqueous emulsion.

In some embodiments, for oral administration, such excipients can comprise pharmaceutical grades of mannitol, lactose, starch, magnesium stearate, sodium saccharine, talcum, cellulose, glucose, gelatin, sucrose, magnesium carbonate, and the like. In some cases, the composition may also contain minor amounts of non-toxic auxiliary substances such as wetting agents, emulsifying agents, or buffers, such a pharmaceutically appropriate: excipient, carrier, diluent, or any combination thereof. In some cases, the composition can be employed in the form of solid preparations for oral administration, the preparations may be tablets, granules, powders, capsules or the like. In a tablet formulation, the composition can be formulated with additives, e.g., an excipient such as a saccharide or cellulose preparation, a binder such as starch paste or methyl cellulose, a filler, a disintegrator, and other additives typically used in the manufacture of medical preparations.

### Administration

In some cases, a pharmaceutical composition can be administered by a method selected from the group consisting of: local, oral, sublingual, buccal, mucosal, nasal, intravenous, subcutaneous, enteral, intra-arterial, intramuscular, intraperitoneal, epidural, intrathecal, intracerebroventricular, intra-articular, intraosseous infusion, intracardiac, intravitreal, parenteral, vaginal, intracavernous, intravesicle, rectal, topical, transdermal, inhalation, perivascular, ocular, ear canal, and any combination thereof.

In some cases, administration can comprise delivery of a composition. In some cases, delivery can comprise injection, inhalation, catheterization, gastrostomy tube administration, intravenous administration, intraosseous administration, ocular administration, otic administration, topical administration, transdermal administration, oral administration, mucosal administration, rectal administration, nasal administration, intravaginal administration, intracavernous administration, intracerebral administration, transurethral administration, buccal administration, sublingual administration, immersion administration, or a combination thereof. A composition provided herein can be administered by any method. Delivery can include topical administration (such as a lotion, a cream, a patch, a gel, a spray, a drip, a liquid formulation, an ointment) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, the subject can administer the composition.

In some embodiments, administering a composition herein can be performed at least about: 1 time per day, 2 times per day, 3 times per day, or more than 4 times per day. In some cases, administering can be performed daily, weekly, monthly, or as needed. In some cases, administration or application of a composition herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In some cases, a treatment duration can be from about: 1 to about 30 days, 1 to about 60 days, 1 to about 90 days, 30 days to about 90 days, 60 days to about 90 days, 30 days to about 180 days, from 90 days to about 180 days, or from 180 days to about 360 days. In some embodiments, administration of a composition disclosed herein can be performed for a treatment duration of at least about 1 week, at least about 2 weeks, at least about 3 weeks, at least about 4 weeks, at least about 1 month, at least about 3 months, at least about 6 moths, at least about 12 months, at least about 1 year or for life. Administration can be performed repeatedly over a lifetime of a subject, such as once a month or once a year for the lifetime of a subject.

In some embodiments, the composition to be administered can contain a quantity of the selected compound in a pharmaceutically effective amount for therapeutic use in a biological system, including a patient or subject according to the present disclosure.

In some embodiments, methods of treating patients or subjects for a particular disease state or infection can comprise administration an effective amount of a pharmaceutical composition comprising a therapeutic engineered MANF protein or functional fragment thereof, a nucleic acid, or a plasmid and/or at least one additional bioactive (e.g., antiviral) agent according to the present disclosure. In some cases, the therapeutically effective amount of a composition herein ranges from about 0.000001 mg/kg to about 1000 mg/kg, wherein mg can be mg of the composition and kg can be kg of bodyweight of the subject. For example, a composition of about: 0.000001 mg/kg, 0.00001 mg/kg, 0.0001 mg/kg, 0.001 mg/kg, 0.01 mg/kg, 0.1 mg/kg. 1.0 mg/kg, 10 mg/kg, 100 mg/kg or 1000 mg/kg can be administered to a subject in need thereof.

The foregoing descriptions of specific embodiments of the present disclosure have been presented for purposes of illustration and description. They are not intended to be exhaustive or to limit the present disclosure to the precise forms disclosed, and many modifications and variations are possible in light of the above teaching. The embodiments were chosen and described to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the scope of the present disclosure.

1. An engineered Mesencephalic Astrocyte-derived Neurotrophic Factor (MANF) protein, a functional fragment thereof, or a nucleic acid encoding the engineered MANF protein or the functional fragment thereof.

2. The engineered MANF protein or the functional fragment thereof of claim 1, wherein the engineered MANF protein or the functional fragment thereof has at least about 80%, about 85%, about 90%, about 95%, about 98% homology or is identical to SEQ ID NO: 2.

3. The engineered MANF protein or the functional fragment thereof of claim 1, wherein the nucleic acid encoding the engineered MANF protein or the functional fragment thereof has at least about 80%, about 85%, about 90%, about 95%, about 98% identity or is identical to SEQ ID NO: 1.

4. The engineered MANF protein or the functional fragment thereof of claim 1, wherein the nucleic acid encoding the engineered MANF protein or the functional fragment thereof further comprises a modified nucleotide.

5. The engineered MANF protein or the functional fragment thereof of claim 4, wherein the modified nucleotide comprises at least one of: a peptide nucleic acid, a morpholino, a phosphorothioate linkage, a locked nucleic acid, a glycol nucleic acid, an additional functional group comprising an amino (—NH2), fluoro (—F), or O-methyl (—OCH3) in a 2′-position of ribose sugar, a threose nucleic acid, a hexitol nucleic acid, a 2′-position sugar modification, a 5-position pyrimidine modification, an 8-position purine modification, a modification at exocyclic amine, a substitution of 4-thiouridine, a 5-((3-indolyl)propionamide-N-allyl)-20-deoxyuridine, a substitution of 5-bromo or 5-iodo-uracil as well as backbone modifications, an anti-reverse cap analog, a substitution of pseudouridine, a 5-methylcytidine and/or N1-methyluridine, or a methylation and unusual base-pairing combinations comprising isobases: isocytidine and isoguanidine and (7-(2-thienyl)imidazo[4,5-b]pyridine, Ds).

6. The engineered MANF protein or the functional fragment thereof of claim 1, wherein the engineered MANF protein or the functional fragment thereof further comprises an epitope-tag fused at a C-terminus of the engineered MANF protein, the functional fragment thereof, or the nucleic acid encoding the engineered MANF protein or the functional fragment thereof further comprises a sequence encoding the epitope-tag.

7. The engineered MANF protein or the functional fragment thereof of claim 6, wherein the epitope-tag is an ALFA-tag, an AViTag, a C-tag, a Calmodulin-tag, an intein capture tag, a polyglutamate tag, a polyarginine tag, an E-tag, a FLAG-tag, an HA-tag, a His-tag, a Gly-His-tag, a Myc-tag, an NE-tag, a Rho1D4-tag, a S-tag, a SBP-tag, a softag 1, a softag 3, a spot-tag, a strep-tag, a T7-tag, a TC tag, a Ty tag, a V5 tag, a VSV-tag, or an Xpress tag.

8. A vector or a plasmid comprising the nucleic acid encoding the engineered MANF protein or the functional fragment thereof of claim 1.

9. The vector or the plasmid of claim 8, wherein the vector is a viral vector.

10. The vector or the plasmid of claim 9, wherein the viral vector is a lentiviral vector or an AAV vector.

11. A composition comprising the engineered MANF protein, the functional fragment thereof of claim 1, and one or more: adjuvant, excipient, carrier, and/or diluent.

12. A pharmaceutical composition comprising the engineered MANF protein, the functional fragment thereof, of claim 1, and one or more: pharmaceutically acceptable adjuvant, pharmaceutically acceptable excipient, pharmaceutically acceptable carrier, and/or pharmaceutically acceptable diluent.

13. The pharmaceutical composition of claim 12, wherein the pharmaceutical composition is in unit dose form.

14. A method for treating a disease or condition arising during or after an infection from an infectious disease or after a vaccination in a subject in need thereof, the method comprising:
administering to the subject in need thereof a therapeutically effective amount of the engineered MANF protein, the functional fragment thereof or the nucleic acid encoding the engineered MANF protein or the functional fragment thereof of claim 1.

15. The method of claim 14, wherein the administrating is by an intravenous injection, an intramuscular injection, a subcutaneous injection, or an intradermal injection.

16. The method of claim 14, wherein the disease comprises a coronavirus.

17. The method of claim 16, wherein the coronavirus is a SARS-COV-2 virus, an evolved variant thereof, or a mutant thereof.

18. The method of claim 14, wherein the administering increases a B-lymphocyte cell population in the subject having a SARS-COV-2 infection compared to a subject having a SARS-COV-2 infection without the administering as measured in an in vitro or an in vivo assay.

19. The method of claim 14, wherein the treating reduces, ameliorates, or improves at least one condition arising during or after the infection from the infectious disease or after the vaccination.

20. The method claim 14, wherein the condition comprises one or more of: long-term breathing problems, heart complications, a chronic kidney impairment, a stroke, a Guillain-Barre syndrome, a multisystem inflammatory syndrome, a neurological damage, a vascular damage, an organ damage, or a clotting.


SEQUENCE LISTING (2 sequences)

SEQ ID NO: 1 - DNA, 1103 bp, synthetic construct
  source 1..1103: mol_type = other DNA
cttcggtcctgctgtagtgccttctgcgccaggcccggttcaatcagcggccacaactgtctagggctcagacaccaccagccaatgagggagggcacgtggagccgcgtctgggctcgcggctcctgaccaatggggaagtggcatgtgggagggcgccggggttccccccgccaatggggagctacggcgcgcggccgggacttggaggcggtgcggcgcggcgggtgcggttcagtcggtcggcggcggcagcggaggaggaggaggaggaggaggatgaggaggatgaggaggatgtgggccacgcaggggctggcggtgcgcgtggctctgagcgtgctgccgggcagccgggcgctgcggccgggcgactgcgaagtttgtatttcttatctgggaagattttaccaggacctcaaagacagagatgtcacattctcaccagccactattgaaaacgaacttataaagttctgccgggaagcaagaggcaaagagaatcggttgtgctactatatcggggccacagatgatgcagccaccaaaatcatcaatgaggtatcaaagcctctggcccaccacatccctgtggagaagatctgtgagaagcttaagaagaaggacagccagatatgtgagcttaagtatgacaagcagatcgacctgagcacagtggacctgaagaagctccgagttaaagagctgaagaagattctggatgactggggggagacatgcaaaggctgtgcagaaaagtctgactacatccggaagataaatgaactgatgcctaaatatgcccccaaggcagccagtgcaccgaccgatttgtagtctgctcaatctctgttgcacctgagggggaaaaaacagttcaactgcttactcccaaaacagcctttttgtaatttattttttaagtgggctcctgacaatactgtatcagatgtgaagcctggagctttcctgatgatgctggccctacagtacccccatgaggggattcccttccttctgttgctggtgtactctaggacttcaaagtgtgtctgggatttttttattaaagaaaaaaaatttctagctgtcaaaaaaaaaaa

SEQ ID NO: 2 - protein, 234 aa, synthetic construct
MGKWHVGGRRGSPRQWGATARGRDLEAVRRGGCGSVGRRRQRRRRRRRRMRRMRRMWATQGLAVRVALSVLPGSRALRPGDCEVCISYLGRFYQDLKDRDVTFSPATIENELIKFCREARGKENRLCYYIGATDDAATKIINEVSKPLAHHIPVEKICEKLKKKDSQICELKYDKQIDLSTVDLKKLRVKELKKILDDWGETCKGCAEKSDYIRKINELMPKYAPKAASAPTDL